(19) United States
(12) Patent Application Publication
(10) Pub. No.: US 2015/0367041 A1
(43) Pub. Date: Dec. 24, 2015

(54) METHOD FOR MANUFACTURING NANO-HYDROXYAPATITE/POLY(LACTIC ACID) HYBRID NANO-COMPOSITE MEMBRANE MAT, NANO-HYDROXYAPATITE/POLY(LACTIC ACID) HYBRID NANO-COMPOSITE MEMBRANE MAT MANUFACTURED THEREBY, AND AIR-JET SPINNING APPARATUS THEREFOR

(71) Applicant: INDUSTRIAL COOPERATION FOUNDATION CHONBUK NATIONAL UNIVERSITY, Jeollabuk-do (KR)

(72) Inventors: Jae Kyoo LIM, Jeollabuk-do (KR); Abdalla ABDALHAY, Jeollbuk-do (KR)

(73) Assignee: INDUSTRIAL COOPERATION FOUNDATION CHONBUK NATIONAL UNIVERSITY, Jeollabuk-do (KR)

(21) Appl. No.: 14/761,633

(22) PCT Filed: Jan. 13, 2014
(86) PCT No.: PCT/KR2014/000352

§ 371 Date: Jul. 17, 2015

(30) Foreign Application Priority Data
Jan. 18, 2013 (KR) 10-2013-0005890

(51) Int. Cl. A61L 31/12; B05D 1/12

(52) U.S. Cl. CPC A61L 31/125; B05D 1/12

(57) ABSTRACT

Provided is a preparing method of a nano hydroxyapatite/poly(lactic acid) hybrid nanocomposite membrane mat including: mixing a hydroxyapatite powder with poly(lactic acid) in a solvent; and preparing a hydroxyapatite/poly(lactic acid) composite mat on a substrate by nozzle-injecting the mixture obtained in the mixing by an air-jet spinning method.

### TECHNICAL FIELD

The present invention relates to a preparing method of a nano hydroxyapatite/poly(lactic acid) hybrid nanocomposite membrane mat, a nano hydroxyapatite/poly(lactic acid) hybrid nanocomposite membrane mat prepared by the same, and an air-jet spinning apparatus for the same, and more particularly, to a preparing method of nano hydroxyapatite/poly(lactic acid) hybrid nanocomposite membrane mat, a nano hydroxyapatite/poly(lactic acid) hybrid nanocomposite membrane mat prepared by the same, and an air-jet spinning apparatus for the same in which large-area coating can be performed on various substrates by using an air-jet spray spinning technique.

### BACKGROUND ART

Techniques of coupling an inorganic reinforcing material with a polymer scaffold have been researched as biological activity and a biological function of a polymer material (C. Huang, R. Chen, Q. Ke, Y. Morsi, K. Zhang, X. Mo, Colloids Surf B Biointerface 82 (2011) 3-7).

Unlike a natural biopolymer, a pure synthetic polymer has low cell affinity due to insufficient cell recognition sites on a scaffold surface. Accordingly, a scaffolding material of a bone tissue needs to have bone conductivity and bone progenitor cells may be absorbed and moved on the scaffold, and as a result, a new bone tissue may be prepared after completely decomposing a temporary 3D scaffold. The present invention provides a preparing method of a unique and effective multi-functional 3D scaffold material by mixing an organic material and an inorganic material by a specific method.

Particularly, in the related art, as an organic/inorganic composite having bioactivity, the composite which is inter-connected with a porous structure has been not yet disclosed, and the effective inter-connection with the porous structure is very important in application of the scaffold composite.

### DETAILED DESCRIPTION OF THE INVENTION

### Technical Problem

The present invention has been made in an effort to provide a preparing method of nano hydroxyapatite/poly(lactic acid) hybrid nanocomposite membrane mat, a nano hydroxyapatite/poly(lactic acid) hybrid nanocomposite membrane mat prepared by the same, and an air-jet spinning apparatus for the same.

### Technical Solution

An embodiment of the present invention provides a preparing method of a nano hydroxyapatite/poly(lactic acid) hybrid nanocomposite membrane mat including: mixing a hydroxyapatite powder with poly(lactic acid) in a solvent; and preparing a hydroxyapatite/poly(lactic acid) composite mat on a substrate by nozzle-injecting the mixture obtained in the mixing by an air-jet spinning method.

The solvent may have a boiling point of 40° C. or less.

The distance between the substrate and the nozzle in which the nozzle-injection is performed may be 50 to 60 cm.

The solvent may be dichloromethane.

The preparing of the composite mat may be performed at relative humidity of 60%

Another embodiment of the present invention provides an air-jet spinning apparatus for the preparing method of the nano hydroxyapatite/poly(lactic acid) hybrid nanocomposite membrane mat.

A distance between a nozzle of the air-jet apparatus and the hybrid nanocomposite membrane mat may be 50 to 60 cm.

Yet another embodiment of the present invention provides a nano hydroxyapatite/poly(lactic acid) hybrid nanocomposite membrane mat prepared by the preparing method of the nano hydroxyapatite/poly(lactic acid) hybrid nanocomposite membrane mat.

The nano hydroxyapatite/poly(lactic acid) hybrid nanocomposite membrane mat may have a structure in which hydroxyapatite nanoparticles are bonded on the surface of the poly(lactic acid) fiber.

Hydrophilicity of the nanocomposite membrane mat may be increased by bonding the hydroxyapatite nanoparticles.

### Advantageous Effects

According to the embodiment of the present invention, large-area coating can be performed on various substrates by using an air-jet spray spinning technique. Particularly, an organic/inorganic hybrid nanocomposite membrane mat was first prepared as an inter-connected porous structure by using the air-jet spinning (AJS) technique. There is an advantage in that the air-jet spinning (AJS) technique according to the present invention may be operated at low cost without using a large apparatus.

### BRIEF DESCRIPTION OF THE DRAWINGS

FIG. 1 is a schematic diagram of a preparing method of a nanocomposite mat by an air-jet spinning method according to an embodiment of the present invention.

FIG. 2 is an SEM image of fibers prepared according to the embodiment of the present invention.

### BEST MODE

Hereinafter, an element separating method according to each embodiment of the present invention will be described with reference to the accompanying drawings.

The following embodiment is a detailed description for assisting understanding of the present invention and it is natural to limit the claims of the present invention. Therefore, the identical invention that performs the same function as the present invention will be included in the scope of the present invention.

When reference numerals refer to components of each drawing, it is noted that although the same components are illustrated in different drawings, the same components are referred to by the same reference numerals as possible. In addition, in the description of the present invention, the detailed descriptions of known related constitutions or functions thereof may be omitted if they make the gist of the present invention unclear.

Further, in describing components of the embodiment of the present invention, terms such as first, second, A, B, (a), (b), and the like may be used. Such a term is only for discriminating the constituent element from another constituent element, and does not limit the essential feature, order, or sequence of the constituent element, or the like. If it is described that a component is “connected”, “coupled” to , or “accesses” the other component, it is understood that the component is directly connected to or accesses the other component but another component may be “connected”, “coupled”, or “access” between the components.

According to the present invention, in order to solve the aforementioned problems in the related art, large-area coating can be performed on various substrates by using an air-jet spray spinning technique. Particularly, an organic/inorganic hybrid nanocomposite membrane mat was first prepared as an inter-connected porous structure by using the air-jet spinning (AJS) technique. There is an advantage in that the air-jet spinning (AJS) technique according to the present invention can be operated at low cost without using a large apparatus.

According to an embodiment of the present invention, pressurized gas is dispersed at a high speed and a polymer solution is increased, and as a result, thin fibers are completed in a nozzle. Simultaneously, the thin fibers prepared in the nozzle are coated on a substrate by evaporating a solvent.

Meanwhile, nanohydroxyapatite (nHA; Ca₁₀(PO₄)₆(OH)₂) is referred to as an ideal bioactive material, and the nanohydroxyapatite has a characteristic of being bonded to the bone under an in-vivo condition. However, in spite of the advantage, nanohydroxyapatite monoliths is weak and has very low resistance to applied loading impact, and may be failed during repeated loading. Further, it is very difficult to be inter-connected with a 3D porous structure, and as a result, it is difficult to use the nanohydroxyapatite in regeneration of a bony lesion.

Active researches on poly(lactic acid) (PLA) have been conducted due to biocompatibility and a reabsorable property. The PLA is used as a suturing material and also used as a rebonding material of the bone and a drug delivery material. However, there is a problem in that these kinds of polymer materials are degraded as acidic materials in an intermediate process, and as a result, there is a problem in that a local inflammatory reaction is caused at an implant site.

Accordingly, in order to overcome the disadvantages of these materials, a HA/PLA composite scaffold for engineering a bone tissue which is synthesized from HA and PLA is prepared according to the present invention. Therefore, in the present invention, a composite scaffold is prepared in accordance with an interaction mechanism between nHA and the polymer material by using the AJS technique.

Hereinafter, the preparing method of the nano hydroxyapatite/poly(lactic acid) hybrid nanocomposite membrane mat according to the embodiment of the present invention will be described in detail.

### Preparation of HA Nano Powder

A HA nano powder was prepared by the following method according to a wet chemical precipitation method without using a surfactant.

First, 0.6M (NH₄)₂HPO₄ aqueous solution was mixed in a 1M Ca(NO₃)₂.4H₂O solution at a velocity of 0.4 ml per minute, and in this case, a Ca/P ratio of the solution was 1.67. Further, pH of the solution was adjusted as 11.

The solution obtained by the mixing was left for 3 hours at room temperature, continuously stirred for 1.5 hours, and heated up to 90° C. Thereafter, the obtained precipitate was stirred for 24 hours to prepare a uniform solution. Finally, the solution was filtered to prepare HA nanoparticles, the prepared HA nanoparticles were dispersed in an alcohol solution to prevent coagulation and then dried to finally prepare the HA nano powder.

### Preparation of nHA/PLA Hybrid Composite Scaffold

First, PLA was dissolved in DCM to prepare PLA solutions having different concentrations of 3 and 7 wt/wt and the solutions were called L-PLA and H-PLA, respectively. Thereafter, the prepared nHA powder was stirred and mixed in the PLA solution for 24 hours and an obtained colloid solution was ultrasonic-treated for 1 hour to prepare a suspension. Further, two colloid solutions were prepared by using nHAs having different amounts of 0.1 g and 0.3 g in a H-PLA solution to be called H-PLA/HA_{0.1} and H-PLA/HA_{0.3}, respectively.

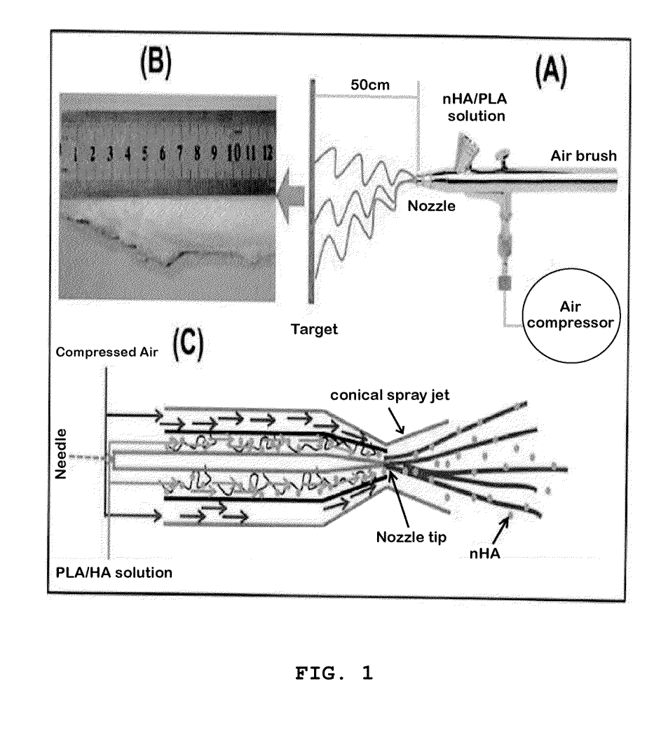

The colloid solution was coated on a substrate by an air-jet spray method to prepare the nHA/PLA hybrid composite scaffold. In the embodiment of the present invention, in the air-jet spray, the colloid solution was sprayed with a nozzle having a diameter of 250 μm by using an air brush spray apparatus (Pa-201, IWATEC, Taiwan, see FIG. 1) to be coated at room temperature, and at the time, humidity was 55%.

Further, air pressure during coating was 420 kPa, and a distance between the substrate (a collector) where the mat was to be formed and an AJS nozzle was 50 to 60 cm.

The inventor found that inter-connected fibers with a few beads were prepared while changing the distance, particularly, in the range. In the embodiment of the present invention, the air brush was fixed in a spray process, and the diameter was covered by a conical spray jet on the substrate in a deposition process.

Thereafter, the prepared material was dried at 37° C. for 24 hours in a vacuum oven to finally prepare the nano hydroxyapatite/poly(lactic acid) hybrid nanocomposite membrane mat.

### Result and Analysis

In the present invention, particularly, solvent evaporation was facilitated in the AJS process by using a solvent having a relatively low boiling point of 40° C. or less. Accordingly, in the present invention, the use of the DCM played an important role in formation of a membrane-shape structure on the fiber. In an experiment example according to the present invention, the entire amount of the entire polymer blend which was transferred to the surface was equalized by using different PLA solutions and controlling a spray (5 ml per minute) duration time.

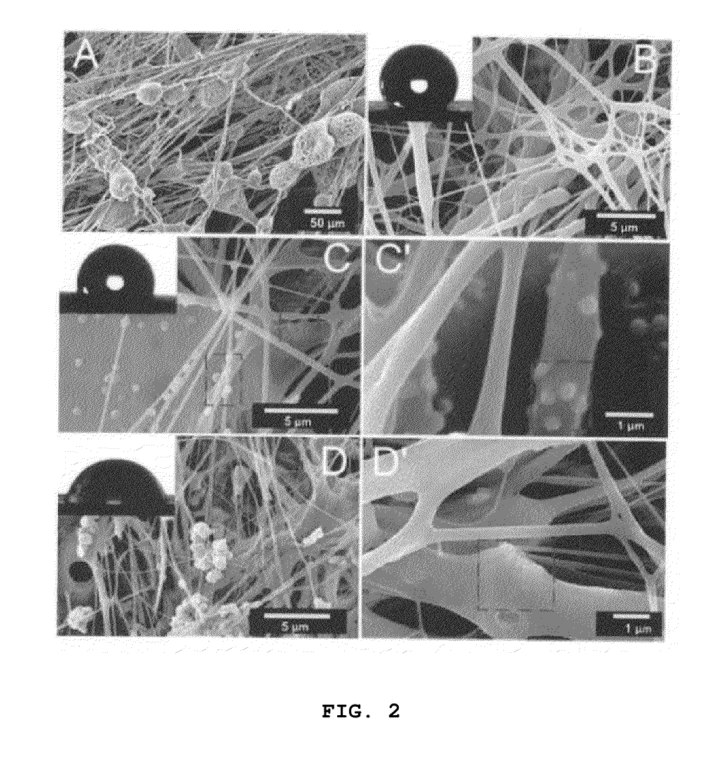

FIG. 2 is an SEM image of fibers manufactured according to the embodiment of the present invention. In FIG. 2, FIG. 2A illustrates L-PLA, FIG. 2B illustrates H-PLA, FIG. 2C illustrates H-PLA/nHA_{0.1}, and FIGS. D and D′ illustrate H-PLA/nHA₁₀.

The inventor found that deformation of a bead structure formed on a line having a relatively low solute concentration of 3% occurred, and each individual fiber strand has a diameter of about 1 to 10 μm and the bead has a diameter of 10 to 40 μm (see FIG. 2A). In addition, a bundle formed by strands contains beads and may be formed at high relative humidity. Similarly, a microbead SEM image has a nano surface characteristic obtained form high relative humidity of 60% (see FIG. 2A).

At a higher polymer concentration, an inter-connected fiber membrane does not contain the beads, and as a result, a PLA fiber prepared by an air-jet spinning method has an average diameter of 780 nm and the diameter shows a narrow distribution of about 420 to 1,139 nm. Further, the inter-connected fiber membrane has a partially coagulated shape (see FIG. 2B). A high fiber production speed induces an excellent coagulation characteristic with respect to an adjacent point and a cross point.

According to the embodiment of the present invention, nHA is added to the PLA solution to prepare an optimized membrane fiber. A nano composite suspension was coated by a spray method under the same condition as the condition which was used for obtaining the PLA/HA biocomposite scaffold. FIGS. 2C to 2D are photographs of fiber mats at different HA concentrations of 0.1 g and 0.3 g. The composite mat containing HA at a low concentration is first separated to different morphologies having lower membrane loading nanoparticles, and the particles are more exactly loaded on the fiber surface.

Referring to FIG. 2, it can be seen that the HA nanoparticles are efficiently loaded on the fiber surface. Further, in a contact angle image of water inserted to the image, it can be seen that the HA nanoparticles are loaded on the PLA fiber surface and as a result, surface modification is effectively performed. That is, It can be seen that the PLA/HA nanocomposite mat has an improved hydrophilic property. As such, a surface with improved wetness due to the hydrophilic property may induce suppression of cell adhesion and protein absorption.

Unless explicitly described to the contrary, since the word “include”, “constitute” or “have” implies the inclusion of stated elements, it may be analyzed that the word “include”, “constitute” or “have” means not the exclusion of any other elements but further inclusion of any other elements. If it is not contrarily defined, all terms used herein including technological or scientific terms have the same meaning as those generally understood by a person with ordinary skill in the art. Terms defined in generally used dictionary shall be construed that they have meanings matching those in the context of a related art, and shall not be construed in ideal or excessively formal meanings unless they are clearly defined in the present application.

Although an embodiment of the present invention has been described for illustrative purposes, those skilled in the art will appreciate that various modifications, changes and substitutions are possible, without departing from the scope and spirit of the invention. Accordingly, the various embodiments disclosed herein are not intended to limit the technical spirit but describe with the true scope and spirit being indicated by the following claims. The protection scope of the present invention should be construed based on the following appended claims and it should be appreciated that the technical spirit included within the scope equivalent to the claims belongs to the present invention.

### INDUSTRIAL APPLICABILITY

The present invention is applicable in a nanocomposite membrane mat industry which is usable in a bio field and the like, as a technique for producing an organic/inorganic hybrid nanocomposite membrane mat.

1. A preparing method of a nano hydroxyapatite/poly(lactic acid) hybrid nanocomposite membrane mat, comprising:
mixing a hydroxyapatite powder with poly(lactic acid) in a solvent; and
preparing a hydroxyapatite/poly(lactic acid) composite mat on a substrate by nozzle-injecting the mixture obtained in the mixing by an air-jet spinning method.

2. The method of claim 1, wherein the solvent has a boiling point of 40° C. or less.

3. The method of claim 1, wherein a distance between the substrate and the nozzle in which the nozzle-injection is performed is 50 to 60 cm.

4. The method of claim 2, wherein the solvent is dichloromethane.

5. The method of claim 1, wherein the preparing of the composite mat is performed at relative humidity of 60%.

6. An air-jet spinning apparatus for the preparing method of the nano hydroxyapatite/poly(lactic acid) hybrid nanocomposite membrane mat of claim 1.

7. The apparatus of claim 6, wherein a distance between a nozzle of the air-jet apparatus and the hybrid nanocomposite membrane mat is 50 to 60 cm.

8. A nano hydroxyapatite/poly(lactic acid) hybrid nanocomposite membrane mat prepared by the preparing method of the nano hydroxyapatite/poly(lactic acid) hybrid nanocomposite membrane mat of claim 1.

9. The mat of claim 8, wherein the nano hydroxyapatite/poly(lactic acid) hybrid nanocomposite membrane mat has a structure in which hydroxyapatite nanoparticles are bonded on the surface of the poly(lactic acid) fiber.

10. The mat of claim 9, wherein hydrophilicity of the nanocomposite membrane mat is increased by bonding the hydroxyapatite nanoparticles.
